(19) United States
(12) Plant Patent Application Publication
(10) Pub. No.: US 2008/0320620 P1
(43) Pub. Date: Dec. 25, 2008

(54) Nectarine tree named 'Hortarine1'

(75) Inventor: Michael Malone, Havelock North (NZ)

Correspondence Address: GREENLEE WINNER AND SULLIVAN P C, 4875 PEARL EAST CIRCLE, SUITE 200, BOULDER, CO, 80301, US

(73) Assignee: The Horticulture & Food Research Institute of New Zealand Limited, Auckland (NZ)

(21) Appl. No.: 12/214,353

(22) Filed: Jun. 18, 2008

Related U.S. Application Data
(60) Provisional application No. 60/936,286, filed on Jun. 19, 2007.

(51) Int. Cl. A01H 5/00

(52) U.S. Cl. PLT/190

(57) ABSTRACT

A new and distinct variety of nectarine tree (Prunus persica) named ‘Hortarine1’ is described. The variety results from selection among a population of open-pollinated seedlings of the peach variety known as ‘Peacharine’ (not patented). The fruit of this new variety has an attractive appearance characterized by yellow skin color with dark red blush, yellow flesh with no anthocyanin present, and intense flavor. The new variety appears suitable for the fresh fruit market and has been named ‘Hortarine1’.

### CROSS-REFERENCE TO RELATED APPLICATION

This application claims the benefit of U.S. provisional application No. 60/936,286 filed 19 Jun. 2007.

### GENUS AND SPECIES OF PLANT CLAIMED

Prunus persica

### VARIETY DENOMINATION

Hortarine1

### BACKGROUND TO THE INVENTION

The present new and distinct variety was originated from an open pollinated population of seedlings derived from the peach variety ‘Peacharine’ (unpatented). Seedlings were planted at Hawke's Bay, New Zealand in 1998. One seedling was selected in February 2002 and was assigned the breeder code, L35/18 and subsequently named ‘Hortarine1’.

‘Hortarine1’ was first asexually propagated in 2002 by budding onto ‘Golden Queen’ (unpatented) seedlings. Trees were field planted in winter 2003. The resulting plants have propagated true to type, demonstrating that the characteristics of the new variety are stable and are transmitted without change through succeeding generations.

The new variety was budded onto ‘Golden Queen’ peach rootstock. The unique combination of characteristics and distinctive color are true to the original seedling tree and are transmitted through succeeding asexual generations.

### SUMMARY OF THE INVENTION

The new variety was selected from a population of seedlings derived from open-pollination of the peach variety known as ‘Peacharine’ (not patented). The new variety was selected in February 2002 from among plants located on land at Hawke's Bay, New Zealand, and was assigned the breeder code, L35/18. The new variety has since been named ‘Hortarine1’.

The new variety is characterized as follows:

Plant form and vigor: trees show medium vigor with an open and spreading habit; suitable for cultivation as single leader or multi-leader tree

Pollination: self-fertile

Fruiting: fruit commences ripening in mid-February in Hawke's Bay, New Zealand.

Yield: medium

Fruit characteristics: a small to medium size nectarine; round fruit shape; skin color is yellow with dark red blush over-color of up to approximately fifty percent of the surface; flesh color is yellow with no anthocyanin present; clingstone.

Flavor and eating quality: the fruit has strong, aromatic flavor similar to the peach variety ‘Peacharine’ (not patented); non-melting texture.

Geographical adaptation: observations indicate that the variety is suitable for regions where annual chill accumulation is approximately 800 hours.

### BRIEF DESCRIPTION OF THE DRAWINGS

The accompanying photographs show typical specimens of the foliage and the fruit of the new variety as depicted in colors as nearly true as is reasonably possible in a color representation of this type. The photograph in FIG. 1 was taken shortly after harvest when the fruit was at eating maturity.

FIG. 1 shows fruit of the variety ‘Hortarine1’

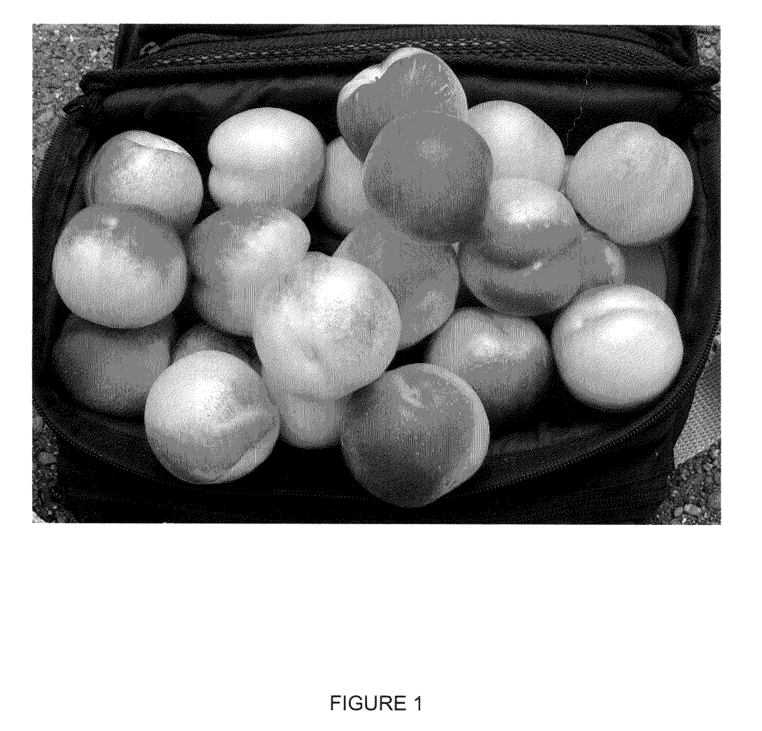

FIG. 2 shows samples of individual fruit of the variety ‘Hortarine1’; view shows blush side, stem end and fruit tip, and fruit suture

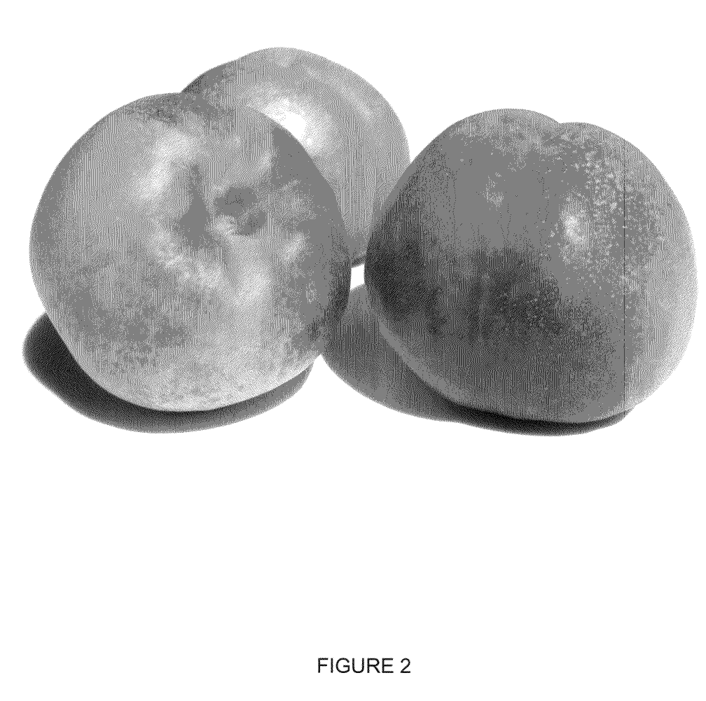

FIG. 3 shows samples of individual fruit of the variety ‘Hortarine1’; view includes cut fruit indicating yellow flesh color with no anthocyanin present.

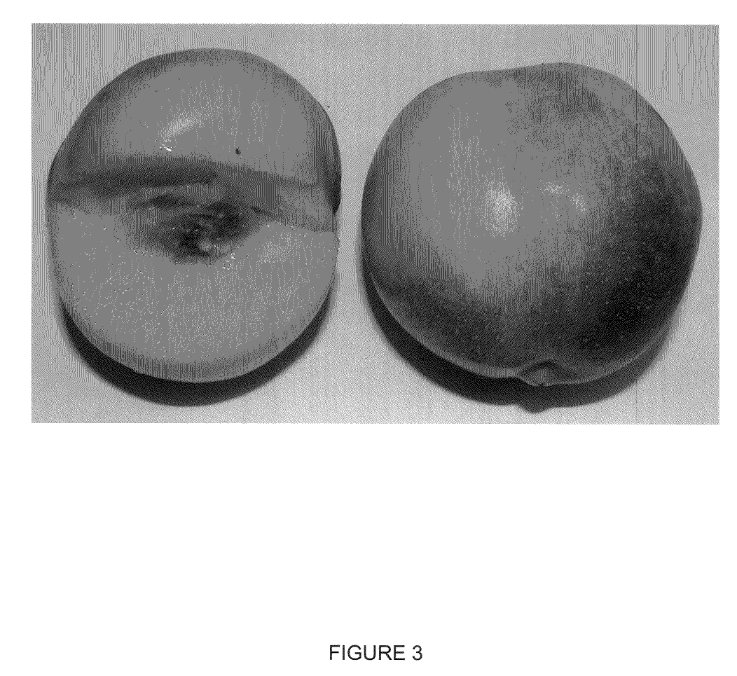

FIG. 4 shows the tree shape and branching development on a 5 year old tree of ‘Hortarine1’ budded on ‘Golden Queen’ seedling rootstock.

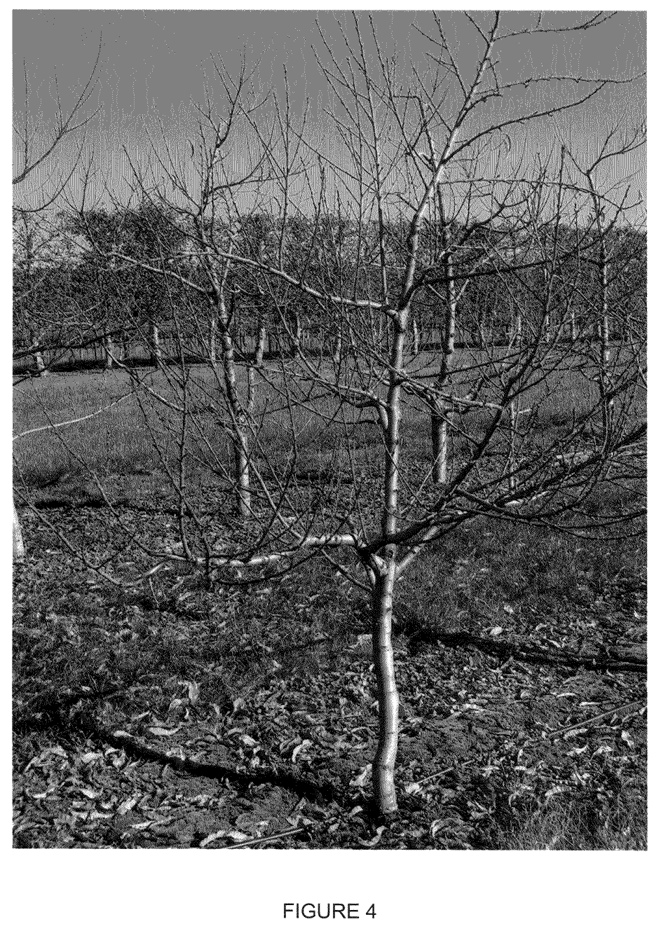

### DETAILED DESCRIPTION OF THE INVENTION

The following is a detailed description of the new variety with color terminology in accordance with The Royal Horticultural Society Color Chart 1986 edition. The specimens described were grown in Havelock North, New Zealand. The observations were made in the 2007 season on trees which were four years old at the time, grown on ‘Golden Queen’ peach rootstock.
Tree:
   Size.—Medium.
   Vigor.—Medium.
   Form.—Pruned to central leader, with a spreading growth habit.
Trunk:
   Size.—Medium.
   Texture.—Smooth.
   Color.—Grey/brown 199B.
Branches:
   Size.—Medium.
   Texture.—Smooth.
   Lenticels.—Medium quantity, medium size.
Leaves:
   Size.—Medium.
   Average width.—35 mm.
   Average length.—116 mm.
   Shape.—Lanceolate.
   Shape of tip.—Acuminate.
   Angle of tip.—Narrow acute.
   Shape of base.—Obtuse.
   Margin.—Serrate.
   Thickness.—Medium.
   Surface.—Smooth.
   Petiole.—Medium.
   Average length.—11.5 mm.
   Average width.—1.8 mm.
   Glands.—Type — Reniform.
   Size.—Medium.
   Average length.—1.1 mm.
   Average width.—0.8 mm.
   Number.—Typically 4 opposite each other in pairs, sometimes 3.
   Location.—Typically on base of leaf blade and the upper end of petiole.
   Color.—upper surface. — Near Dark Green 139A.
   Color.—lower surface. — Near Green 136B.
Flowers:
   Bud size.—Medium.
   Bud length.—Medium.
   Form.—Plump.
   Flower size.—Medium to large.
   Pollen.—Present.
   Color.—Medium pink.
   Petal shape.—Round.
   Pubescence of ovary.—Absent.
Fruit:
   Maturity for consumption.—Firm ripe.
   First picking.—Late-season.
   Size.—Small to medium.
   Average weight.—110 g.
   Average diameter across suture line.—58 mm.
   Average length.—60 mm.
   Shape (ventral view).—Round, symmetrical.
   Shape of pistil end.—strongly pointed.
   Suture.—Medium prominence, extends from base to apex.
Flesh:
   Ripens.—Evenly.
   Texture.—Firm, non-melting canning clingstone.
   Fibers.—Non-fibrous.
   Aroma.—Strong.
   Average soluble solids.—20 Brix.
   Juice.—Low to Moderate.
   Color.—Yellow 9A . Anthocyanin coloration of flesh absent.
Stem:
   Size.—Medium.
Skin:
   Thickness.—Medium.
   Adherence to flesh.—Medium to strong.
   Pubescence.—Nil.
   Tendency to crack.—None.
   Color.—Orange-red N25A blending to red N30B, marbled to flush pattern of over color; Yellow 22D ground color of skin.
Stone:
   Type.—Clingstone.
   Size.—Medium to large.
   Average stone length.—39 mm.
   Average stone width.—25 mm.
   Average stone thickness.—16 mm.
   Average stone weight.—6.9 g.
   Shape (lateral view).—Elliptic.
   Surface.—Pits and grooves.
   Tendency to split.—Very low.
   Color.—Yellow Brown N167B.
   Use.—Local and export markets.
   Storage.—Good; trials indicate 2-3 weeks storage without internal breakdown of flesh.

The present new variety of peach tree, its flowers, foliage and fruit herein described may vary slightly in detail due to climate, soil conditions and cultural practices under which the variety may be grown. The present description is that of the variety grown under the environmental conditions prevailing near Havelock North, Hawke's Bay, New Zealand.

I claim:

1. A new and distinct variety of peach tree named ‘Hortarine1’, as herein illustrated and described.
